(19) United States
(12) Patent Application Publication
(10) Pub. No.: US 2009/0298920 A1
(43) Pub. Date: Dec. 3, 2009

(54) CHIMERIC TRANSFER RNA AND USE THEREOF FOR THE PRODUCTION OF RNA BY A CELL

(75) Inventors: Frédéric Dardel, Paris (FR); Luc Ponchon, Paris (FR)

Correspondence Address: YOUNG & THOMPSON, 209 Madison Street, Suite 500, ALEXANDRIA, VA, 22314, US

(73) Assignee: UNIVERSITE RENE DESCARTES - PARIS V, Paris Cedex 06 (FR)

(21) Appl. No.: 12/304,623

(22) PCT Filed: Jun. 13, 2007
(86) PCT No.: PCT/FR07/00980

§ 371 Date: Jun. 5, 2009

(30) Foreign Application Priority Data
Jun. 14, 2006 (FR) 0605304

(51) Int. Cl. A61K 31/7088; C12P 19/34; C07H 21/02; C12Q 1/68; C12N 15/70; C12N 1/21

(52) U.S. Cl. 514/44 R; 435/91.31; 536/24.1; 536/24.5; 435/6; 435/320.1; 435/252.3

(57) ABSTRACT

The present invention relates to the use of a nucleic acid encoding a chimeric transfer RNA (tRNA), which chimeric tRNA originates from the modification of a tRNA by insertion of an RNA into the stem-loop of the anticodon of the tRNA and/or by substitution of all or part of the stem-loop of the anticodon of the tRNA with an RNA, for the production of the RNA or of a part of the RNA, in a cell.

The present invention relates in particular to the use of a chimeric tRNA comprising a RNA for the production of said RNA by a cell.

At present, the production of RNA in large quantities substantially makes use of three distinct technologies: chemical synthesis, in vitro enzymatic synthesis, and the purification of RNAs produced in vivo, generally in isolated eukaryotic or prokaryotic cells.

Chemical synthesis (Marshal & Kaiser (2004), Curr. Opin. Chem. Biol. 8: 222-229) allows a RNA molecule to be produced in quantities of the order of from 100 μg to 10 mg. That technology is limited, however, to relatively short molecules generally comprising fewer than 50 ribonucleotides, all the more so since the synthesis is carried out on a large scale, i.e. for quantities greater than one milligram. In addition, that technology is relatively expensive.

In vitro enzymatic synthesis enables RNA molecules to be produced using a purified enzyme, a DNA template and ribonucleotide triphosphates (Milligan et al. (1987) Nucleic Acids Res. 15: 8783-8798). Unlike chemical synthesis, there is no limit in respect of the size of the synthesized molecules. However, its use remains tricky for large amounts, with production yields that are very variable and highly dependent on the sequence of the RNA molecules to be produced. In addition, purification is found to be laborious and especially requires multiple electrophoreses and electroelutions. That technology is also relatively expensive.

Finally, the purification of RNAs produced in vivo principally makes it possible to obtain RNA molecules that are produced naturally by the cells and are relatively abundant, such as tRNAs (Meinnel et al. (1988) Nucl. Acids Res. 16: 8095-8096; Normanly et al. (1986) Proc. Natl. Acad. Sci. 83: 6548-6552; Tisne et al. (2000) RNA 6: 1403-1412), the ribonucleotide part of ribonuclease P (Meinnel & Blanquet (1995) J. Biol. Chem. 270: 15908-15914) or tmRNA (Gaudin et al. (2003) J. Mol. Biol. 331: 457-471). That technology has never been applied systematically to RNAs other than natural RNAs, especially owing to foreseeable considerable technical obstacles such as the instability of the RNAs produced, the low expression yield or difficulties with purification.

An object of the invention is, therefore, to provide a means of producing RNA that does not have the disadvantages encountered with the technologies mentioned above.

tRNAs are fundamental molecules of peptide biosynthesis which, once loaded with their respective amino acids by the aminoacyl tRNA synthetases, effect, thanks to the ribosome, the translation of the genetic message carried by the messenger RNAs into peptide sequences (Hopper & Phisicky (2003), Genes dev. 17:162-180).

The document Medina & Joshi (1999) Nucl. Acids Res. 27: 1698-1708 describes a chimeric tRNA in which a ribozyme is inserted between two successive bases of the loop of the anticodon of human tRNA^{Lys}₃. However, that chimeric tRNA is produced in vitro with the disadvantages inherent in that technology that have been mentioned above.

The present invention results from the unexpected finding that it is possible to produce large quantities of a RNA molecule from cells that express a modified tRNA, for example in such a manner that part of the stem-loop of the anticodon is replaced by the coding sequence of the RNA molecule. The modified tRNA containing the RNA molecule is readily purified, in a large quantity, starting from the cells, it being possible for the RNA molecule to be produced to be subsequently excised from the chimeric tRNA.

The present invention accordingly relates to the use of a nucleic acid coding for a chimeric transfer RNA (tRNA), which chimeric tRNA is derived from the modification of a tRNA by insertion of a RNA into the stem-loop of the anticodon of said tRNA and/or by substitution of all or part of the stem-loop of the anticodon of said tRNA with a RNA, for the production of said RNA, or of part of said RNA, in a cell.

As it is understood here, the term “production” of the RNA refers to the production of the RNA in itself, or of part of this RNA, but also to the production of the RNA within the chimeric tRNA or part of this chimeric tRNA. Production is preferably effected starting from the transcription of the nucleic acid by the cell. Transcription of the nucleic acid leads to the chimeric tRNA. If necessary, the chimeric tRNA may be cleaved inside or outside the cell in order to release the RNA.

In a particular embodiment of the invention, the RNA defined above substitutes all or part of the stem-loop of the anticodon contained between the first ribonucleotide, inclusive, of the stem-loop of the anticodon and the last ribonucleotide, inclusive, of the stem-loop of the anticodon.

The use according to the invention of a nucleic acid coding for chimeric tRNA as defined above in the production of the RNA is particularly advantageous because the presence of the tRNA part, or tRNA framework, especially allows the RNA (included in the chimeric tRNA) to be produced in a yield greater than that which would be obtained in the absence of the tRNA part, and allows the RNA (included in the chimeric tRNA) to be protected from degradation, especially associated with some cell components.

### tRNA

The general characteristics of a tRNA are well-known to the person skilled in the art.

Preferably, a tRNA is formed of a single ribonucleotide chain which is capable of folding to adopt a characteristic, so-called cloverleaf secondary structure. This characteristic secondary structure comprises:

(i) an acceptor stem composed of the first 7 ribonucleotides of the 5′ end of the ribonucleotide chain and the 7 ribonucleotides that precede the last 4 ribonucleotides of the 3′ end of the ribonucleotide chain, thus forming a double-stranded structure comprising 6 or 7 pairs of ribonucleotides, it being possible for the ribonucleotides constituted by the first ribonucleotide of the 5′ end of the ribonucleotide chain and the ribonucleotide that precedes the last 4 ribonucleotides of the 3′ end of the ribonucleotide chain not to be paired;
(ii) a D arm constituted by 4 pairs of ribonucleotides and a D loop constituted by 8 to 10 ribonucleotides, formed by the folding of a part of the ribonucleotide chain that follows the first 7 ribonucleotides of the 5′ end of the ribonucleotide chain;
(iii) a stem of the anticodon constituted by 5 pairs of ribonucleotides, and a loop of the anticodon constituted by 7 ribonucleotides (stem-loop of the anticodon), formed by the folding of a part of the ribonucleotide chain that follows the D arm and the D loop;
(iv) a variable loop constituted by from 4 to 21 ribonucleotides and formed by a part of the ribonucleotide chain that follows the stem of the anticodon and the loop of the anticodon;
(v) a T arm constituted by 5 pairs of ribonucleotides, and a T loop constituted by 8 ribonucleotides, formed by the folding of a part of the ribonucleotide chain that follows the variable loop and precedes the ribonucleotides of the 3′ end of the ribonucleotide chain which are involved in the constitution of the acceptor stem.

As it is understood here, a pair of ribonucleotides is formed by the non-covalent pairing of the purine and pyrimidine bases of the two ribonucleotides thanks to weak bonds, such as hydrogen bonds, which may especially be Watson-Crick type bonds, which are well-known to the person skilled in the art.

Likewise preferably, from the 5′ end in the direction towards the 3′ end, 2 ribonucleotides are present between the first 7 ribonucleotides of the 5′ end of the ribonucleotide chain and the D arm and loop, 1 ribonucleotide is present between the D arm and loop, on the one hand, and the stem and the loop of the anticodon, on the other hand, and 1 ribonucleotide is present between the stem and the loop of the anticodon, on the one hand, and the variable loop, on the other hand.

Still preferably, and according to the numbering well-known to the person skilled in the art and defined by Sprinzl et al. (1998) “Compilation of tRNA sequences and sequences of tRNA genes”. Nucleic Acids Res. 26: 148-153, the tRNA comprises 17 ribonucleotides, ensuring the three-dimensional structure of the tRNA and recognition by the cell enzymes, namely: U₈, A₁₄, (A or G)₁₅, G₁₈, G₁₉, A₂₁, G₅₃, U₅₄, U₅₅, C₅₆, (A or G)₅₇, A₅₈, (C or U)₆₀, C₆₁, C₇₄, C₇₅, A₇₆. The indicated ribonucleotides correspond to the sequence of the tRNA as transcribed before any post-transcriptional modifications of certain ribonucleotides by the cell machinery.

In particular, the tRNA defined above may be selected from the group constituted by Archean, bacterial, viral, protozoan, fungal, algal, plant or animal tRNAs.

The tRNAs which can be used according to the invention also include all the tRNAs described by Sprinzl et al. (1998) “Compilation of tRNA sequences and sequences of tRNA genes”. Nucleic Acids Res. 26: 148-153 or those available on the site: http://www.uni-bayreuth.de/departments/biochemie/trna/.

In the context of the invention, the term “tRNA” also includes structures obtained by modifying a tRNA as defined above or natural variants of a tRNA as defined above, provided that those modified structures or those variants retain the functionalities of the unmodified tRNA, namely especially the interaction with proteins such as EF-Tu factor (see, for example, Rodnina et al. (2005) FEBS. Lett. 579: 938-942) or CCAse (see, for example, Augustin et al. (2003) J. Mol. Biol. 328: 985-994).

### RNA

The RNA according to the invention is any ribonucleic chain, which preferably comprises from 6 to 5,000 ribonucleotides, more preferably from 6 to 1,000 ribonucleotides and yet more preferably from 6 to 300 ribonucleotides.

In another particular embodiment of the invention, all or part of the RNA defined above is selected from the list constituted by an antisense RNA, an interfering RNA, an aptamer, a ribozyme, a viral RNA, a ribosomal RNA and a nucleolar RNA.

The expression “antisense RNA” denotes a RNA that is capable of binding to a target nucleic sequence (DNA or RNA) so as to limit or prevent its functioning; in particular, antisense RNAs are able to bind to a target messenger RNA in order to prevent its translation (see, for example, Tafech et al. (2006) Curr. Med. Chem. 13: 863-881).

The expression “interfering RNA” denotes a RNA capable of preventing or limiting the expression of a target gene by the phenomenon of interference (see, for example, Tafech et al. (2006) Curr. Med. Chem. 13: 863-881).

The term “aptamer” denotes a RNA capable of binding to a target compound, such as a biological macromolecule, for example of proteinic nature (see, for example, Nimjee et al. (2005) Ann. Rev. Med. 56:555-583).

The term “ribozyme” denotes a RNA capable of catalyzing one or more chemical reactions (see, for example, Fiammenga & Jaschke (2005) Curr. Opin. Biotechnol. 16: 614-621).

The expression “viral RNA” denotes a RNA or part of a RNA carried or encoded by a virus.

The expressions “ribosomal RNA” and “nucleolar RNA” denote a RNA or part of RNA constituting the ribosome or the nucleolus, respectively.

In another particular embodiment of the invention, the RNA is structured.

The expression “structured RNA” denotes a RNA capable of adopting a secondary structure and optionally a preferred tertiary structure.

In another particular embodiment of the invention, the RNA comprises a purification tag, the purification tag preferably being selected from the group constituted by a ribozyme and an aptamer.

The expression “purification tag” denotes a pattern, preferably of ribonucleotide nature, which is capable of promoting the separation, for example separation by affinity, of the chimeric tRNA comprising it, from the medium in which it is located.

The ribozyme is preferably selected from the group constituted by a hairpin ribozyme, a hammerhead ribozyme and a leadzyme (see, for example, Doherty & Doudna (2000) Ann. Rev Biochem. 69: 597-615).

The aptamer is preferably selected from the group constituted by an aptamer that binds to avidin, to dextran (Sephadex™), to biotin or to arginine.

### Chimeric tRNA

When the RNA substitutes all or part of the stem-loop of the anticodon contained between the first ribonucleotide, inclusive, of the stem-loop of the anticodon and the last ribonucleotide, inclusive, of the stem-loop of the anticodon, the chimeric tRNA is preferably such that the two ribonucleotides that follow the ribonucleotide that precedes the stem-loop of the anticodon in the tRNA before modification are paired with the two ribonucleotides that precede the ribonucleotide that follows the stem-loop of the anticodon in the tRNA before modification. Accordingly, in a particular case, the chimeric tRNA is such that the two base pairs of the end of the stem of the anticodon that is directed towards the T arm and the D arm of the tRNA are retained. In another particular case, the chimeric tRNA is such that the first two ribonucleotides of the RNA pair with the last two ribonucleotides of the RNA.

Preferably, the chimeric tRNA defined above preferably has the following formula (I):

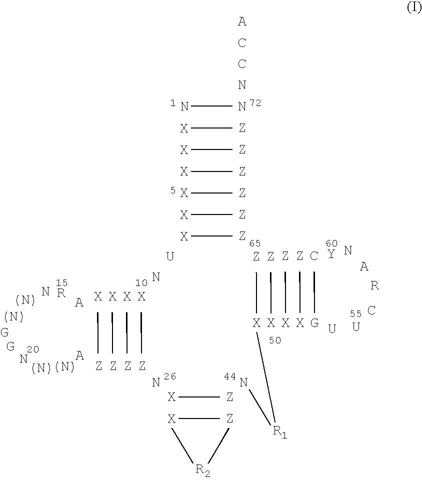

wherein:
A represents adenine or one of its analogs, C represents cytosine or one of its analogs, G represents guanosine or one of its analogs, and U represents uridine or one of its analogs,
each of the N, which may be identical or different, represents any ribonucleotide,
each of the (N), which may be identical or different, represents any ribonucleotide, which may be present or absent,
R represents A or G, or their analogs,
Y represents U or C, or their analogs,
each of the X-Z, which may be identical or different, represents a pair A-U, U-A, G-C, C-G, G-U or U-G, or their analogs,
the ribonucleotides N in position 1 and N in position 72 may or may not be paired,
R₁ represents a sequence of from 3 to 20 ribonucleotides,
R₂ represents the inserted RNA, namely a sequence of from 6 to 5,000 ribonucleotides, more preferably from 6 to 1,000 ribonucleotides and yet more preferably from 6 to 300 ribonucleotides.

As it is understood here, the term “analog” defines possible derivatives of the ribonucleotide originating from the activity of tRNA post-transcriptional modification enzymes of the cell in which they are produced. The analogs of the ribonucleotides A, C, G and U which may be found in a tRNA depend on the cell in which that tRNA is produced and on the position of the ribonucleotide in question in the tRNA. A large number of analogs are given in Sprinzl et al. (1998) “Compilation of tRNA sequences and sequences of tRNA genes”. Nucleic Acids Res., 26, 148-153 and on the basis of “RNA modification database” data (http://medstat.med.utah.edu/RNAmods/). The analogs of A may be selected more particularly from the group constituted by 1-methyl-A, inosine and 2′-O-methyl-A. The analogs of C may be selected more particularly from the group constituted by 5-methyl-C and 2′-O-methyl-C. The analogs of G may be selected more particularly from the group constituted by 7-methyl-G and 2′-O-methyl-G. The analogs of U may be selected more particularly from the group constituted by pseudouridine, ribothymidine, 2′-O-methyl-ribothymidine, dihydrouridine, 4-thiouridine and 3-(3-amino-3-carboxypropyl)-uridine.

In the formula above, which uses the conventions well-known to the person skilled in the art for representing tRNAs, the covalent bonds which join together the ribonucleotides of the ribonucleic chain are not shown and the lines between the ribonucleotides N—N, X-Z and C-G represent weak bonds, preferably of the hydrogen type. By contrast, the lines joining R₂ to X and R₁ to N and X represent covalent bonds.

By way of example, a general representation of a chimeric tRNA according to the invention is thus shown in FIG. 1.

Preferably, the chimeric tRNA defined above has one of the following formulae:

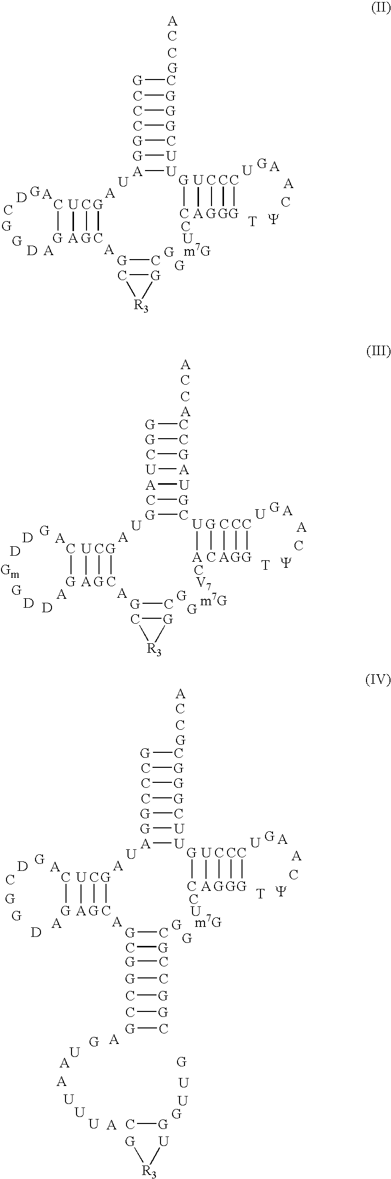

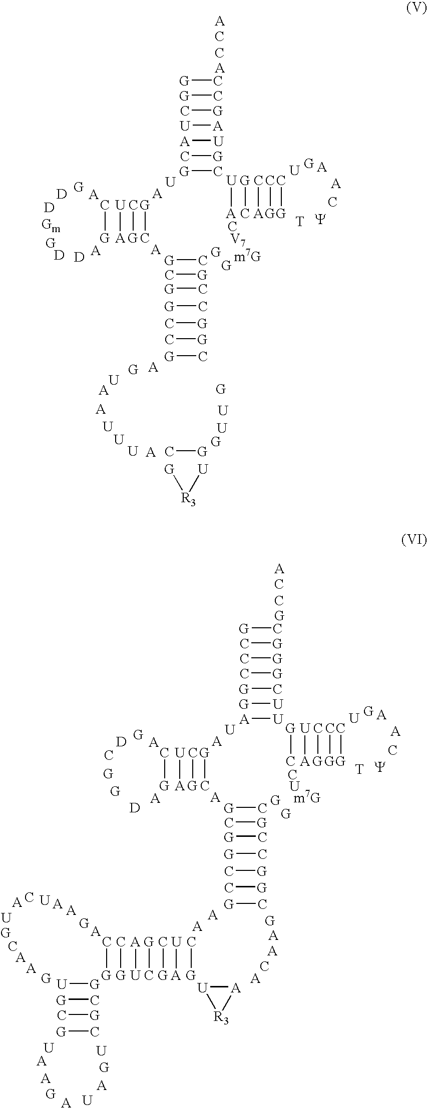

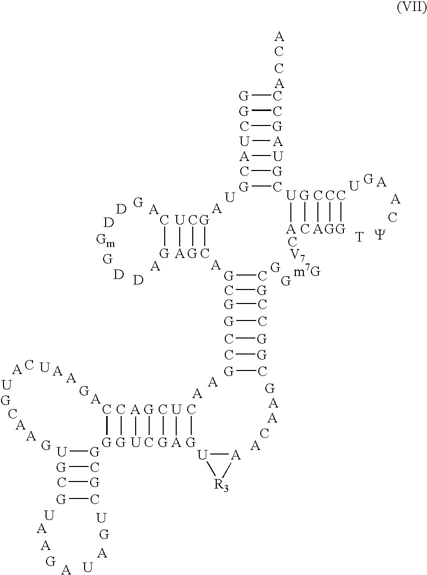

wherein:
D represents dihydrouridine,
Gₘ represents 2′-O-methyl-guanosine,
T represents ribothymidine,
Φ represents pseudouridine,
^{m7}G represents 7-methyl-guanine,
V₇ represents 3-(3-amino-3-carboxypropyl)-uridine,
R₃ represents a sequence of from 6 to 5,000 ribonucleotides, more preferably from 6 to 1,000 ribonucleotides and yet more preferably from 6 to 300 ribonucleotides.

Formulae (II), (IV) and (VI) represent a modified human tRNA^{Lys}₃. Formulae (III), (V) and (VII) represent a modified tRNAₘ^{Met} of E. coli. In formulae (IV) and (V), the tRNA part is bound to an aptamer that binds to dextran (Sephadex™). In formulae (VI) and (VII), the tRNA part is bound to an aptamer that binds to streptavidin. In addition, in formulae (II), (IV) and (VI), the dot between the ribonucleotides G and U of the acceptor stem means that they are bound by way of two hydrogen bonds in a non-Watson-Crick type pairing, this notation being well-known to the person skilled in the art.

In another particular embodiment of the invention, the chimeric tRNA defined above does not comprise the substantially intact stem of the anticodon of the tRNA from which it is derived. This means, especially, that, in the chimeric tRNA, between the ribonucleotide that precedes the stem-loop of the anticodon in the tRNA before modification and the ribonucleotide that follows the stem-loop of the anticodon in the tRNA before modification, the stem of the anticodon of the tRNA before modification is no longer present.

### Cell

The cell in which the tRNA is produced is preferably isolated, especially when it is an animal or human cell. In addition, the cell may be a cell of any type, eukaryotic or prokaryotic. The cell is preferably a cell of the bacterial type. The cell is particularly preferably of the Escherichia coli type.

### Nucleic Acid

In another particular embodiment of the invention, the nucleic acid coding for the chimeric tRNA defined above is a DNA. This DNA is preferably contained in an expression vector comprising a promoter and a terminator, which are operably linked to the nucleic acid, as well as a replication origin and a selection marker.

Preferably, the nucleic acid defined above is introduced into the cell in which it expresses a chimeric tRNA as defined above. The means of introducing and expressing a nucleic acid in a cell are well-known to the person skilled in the art.

The present invention relates also to a chimeric tRNA as defined above.

The present invention relates also to a nucleic acid coding for a chimeric tRNA as defined above.

The present invention relates also to an expression vector comprising a nucleic acid as defined above, a promoter and a terminator, which are operably linked to the nucleic acid, as well as a replication origin and a selection marker.

The present invention relates also to a cell comprising a nucleic acid as defined above or an expression vector as defined above.

The cell is preferably a bacterium, especially of the E. coli type.

The present invention relates also to a second nucleic acid which is suitable for the preparation of a nucleic acid as defined above, and comprising, in the 5′-3′ direction, at least:

(i) a sequence coding for a part of a tRNA extending from the 5′ end of said tRNA to the ribonucleotide that precedes the first ribonucleotide of the stem of the anticodon or to a ribonculeotide of the stem or of the loop of the anticodon;
(ii) optionally a sequence coding for all or part of a purification tag;
(iii) at least one cleavage site of a restriction enzyme;
(iv) optionally a sequence coding for all or part of a purification tag;
(v) a sequence coding for a part of the tRNA extending from a ribonucleotide of the stem or of the loop of the anticodon downstream of the ribonucleotide of (i) or of the ribonucleotide that follows the last ribonucleotide of the stem of the anticodon to the 3′ end of said tRNA,
provided that the sequence of the second nucleic acid in its entirety is not the coding sequence of the tRNA.

Preferably, in the second nucleic acid as defined above, which is suitable for the preparation of a nucleic acid as defined above, the sequence defined in (i) extends from the 5′ end of said tRNA to the second ribonucleotide of the stem of the anticodon, and the sequence defined in (ii) extends from the penultimate ribonucleotide of the stem of the anticodon to the 3′ end of said tRNA.

Preferably, the sequence of the second nucleic acid as defined above, which is suitable for the preparation of a nucleic acid as defined above, is selected from the group constituted by:
SEQ ID NO: 1;
SEQ ID NO: 2;
SEQ ID NO: 3;
SEQ ID NO: 4;
SEQ ID NO: 5;
SEQ ID NO: 6.
   SEQ ID NO: 1 is suitable for the preparation of a chimeric tRNA of formula (II).
   SEQ ID NO: 2 is suitable for the preparation of a chimeric tRNA of formula (III).
   SEQ ID NO: 3 is suitable for the preparation of a chimeric tRNA of formula (IV).
   SEQ ID NO: 4 is suitable for the preparation of a chimeric tRNA of formula (V).
   SEQ ID NO: 5 is suitable for the preparation of a chimeric tRNA of formula (VI).
   SEQ ID NO: 6 is suitable for the preparation of a chimeric tRNA of formula (VII).

The present invention relates also to an expression vector comprising a second nucleic acid as defined above, which is suitable for the preparation of a nucleic acid as defined above, a promoter and a terminator, which are operably linked to the nucleic acid, as well as a replication origin and a selection marker. The sequence of the expression vector is preferably selected from the group constituted by:
SEQ ID NO: 7;
SEQ ID NO: 8;
SEQ ID NO: 9;
SEQ ID NO:10;
SEQ ID NO: 11;
SEQ ID NO:12.
   SEQ ID NO: 7 to 12 comprise SEQ ID NO: 1 to 6, respectively.

In the above vectors, the promoter is preferably selected from the group constituted by the promoters lpp, lac, tac and trc and ara of E. Coli, the promoter pL of the lambda bacteriophage or the promoter of the T7 bacteriophage.

In the above vectors, the terminator is preferably a terminator of ribosomal RNA operons, especially selected from the group constituted by rrnA, rrnB and rrnC.

In the above vectors, the selection marker is preferably an antibiotic resistance gene, especially selected from the group constituted by an ampicillin, kanamycin or chloramphenicol resistance gene.

The present invention relates also to a method for producing a RNA, wherein:
cells transformed by a nucleic acid as defined above are cultivated;
the chimeric tRNA is recovered from the cultivated cells or from the culture supernatant of the cultivated cells;
the chimeric tRNA is optionally cleaved in order to recover the RNA to be produced in isolated form.

The present invention relates also to a kit for the production of a RNA with the aid of a chimeric tRNA comprising it, which kit comprises at least:
an expression vector comprising a second nucleic acid as defined;
a means for cleaving a chimeric tRNA allowing to release the RNA to be produced;
optionally at least one restriction enzyme that cleaves at the restriction site defined above;
optionally cells capable of being transformed by a nucleic acid and of producing a chimeric tRNA.

The above kit may also comprise a purification ligand that binds to the purification tag contained, where appropriate, in the chimeric tRNA.

In a particular embodiment of the kit defined above, the expression vector is a bacterial plasmid and the cells are bacteria.

In another particular embodiment of the kit as defined above, the cleavage means is constituted by RNase H and by two oligonucleotides that are complementary, respectively, to a part of the sequence of the chimeric tRNA that precedes the 5′ end of the RNA to be produced, and to a part of the sequence of the chimeric tRNA that follows the 3′ end of the RNA to be produced.

The RNase advantageously degrades the oligonucleotide-RNA hybrids, which releases the RNA.

In a preferred embodiment of the kit as defined above:
the bacteria are of the Escherichia coli type;
the expression vector is represented by SEQ ID NO: 7 and the oligonucleotides are represented by SEQ ID NO: 13 and SEQ ID NO: 14, or
the expression vector is represented by SEQ ID NO: 8 and the oligonucleotides are represented by SEQ ID NO: 15 and SEQ ID NO: 16, or
the expression vector is represented by SEQ ID NO: 9 and the oligonucleotides are represented by SEQ ID NO: 13 and SEQ ID NO: 14, or
the expression vector is represented by SEQ ID NO: 10 and the oligonucleotides are represented by SEQ ID NO: 15 and SEQ ID NO: 16, or
the expression vector is represented by SEQ ID NO: 11 and the oligonucleotides are represented by SEQ ID NO: 13 and SEQ ID NO: 14, or
the expression vector is represented by SEQ ID NO: 12 and the oligonucleotides are represented by SEQ ID NO: 15 and SEQ ID NO: 16.

The invention relates also to the use of a chimeric tRNA as defined above for resolving the three-dimensional structure of the inserted or substituted RNA, by applying the technique of nuclear magnetic resonance to a solution of the chimeric tRNA or by applying the technique of X-ray diffraction to crystals of the chimeric tRNA.

Indeed, the structure of the inserted or substituted RNA is advantageously retained in the chimeric tRNA relative to the RNA in isolated form. Moreover, in the context of crystallography, the presence of the tRNA part may improve crystallization of the chimeric tRNA in its entirety. In addition, if a tRNA of known crystallographic structure is used for the production of the chimeric tRNA, the crystallographic structure data of the tRNA can then be used for the resolution of the crystallographic structure of the chimeric tRNA in its entirety, especially in the step of framing or molecular replacement.

The invention relates also to the in vitro, ex vivo or in vivo use of a chimeric tRNA as defined above as an antisense RNA, an interfering RNA, an aptamer or a ribozyme when the inserted or substituted RNA is an antisense RNA, an interfering RNA, an aptamer or a ribozyme, respectively.

Indeed, advantageously and where appropriate, the chimeric tRNAs of the invention are such that the activity of the inserted or substituted RNA is retained relative to the RNA in isolated form.

The present invention relates also to a pharmaceutical composition comprising a chimeric tRNA as defined above as active ingredient, in association with a pharmaceutically acceptable carrier.

In addition, the chimeric tRNAs according to the invention may be used to produce combinatorial libraries of RNA, especially with the aid of RNA molecules obtained in a combinatory manner. These combinatorial libraries of RNA may be used in order to screen potential pharmacological targets. A contrario, chimeric tRNAs according to the invention may be screened, especially when they express RNAs which are potential pharmacological targets, such as bacterial ribosomal RNAs or viral RNAs, with the aid of candidate medicaments.

Finally, the expression of chimeric tRNAs within cells makes it possible to envisage co-purification with partners of the RNAs, such as, for example, direct purification of ribonucleoprotein complexes, which would permit especially the identification of partners, proteic or otherwise, of a given RNA.

The invention will be illustrated further with the aid of the following examples, which are not limiting.

### DESCRIPTION OF THE FIGURES

FIG. 1

FIG. 1 shows the structure of a chimeric tRNA according to the invention. The retained part of the tRNA is called the tRNA framework. The nucleotides shown in brackets are optional. The nucleotides shown in bold type and in gray type correspond to the conserved or semi-conserved positions.

FIG. 2

FIG. 2 shows the structure of a chimeric tRNA^{Lys}₃ incorporating the epsilon domain of the human hepatitis B virus.

FIG. 3

FIG. 3 shows the structure of a chimeric tRNA^{Lys}₃ incorporating the epsilon domain of the human hepatitis B virus (on the left) and the corresponding HSQC spectrum (on the right). The spectrum was recorded at 15° C. on a 600 MHz Bruker Avance spectrometer. The chimeric RNA was dialyzed against distilled water and then lyophilized and finally dissolved in a 90% H₂O/10% D₂O mixture at a concentration of 1 mmol/liter (total volume approximately 400 μl). The spectral region shown corresponds to the displacements (vertical and horizontal axes, ppm) of the NH imino groups involved in the base pairings. A given peak corresponds to each A-U or G-C pairing in the RNA. This spectrum is a “signature” of the 2D and 3D structure of the studied RNA. It shows the peaks corresponding to the tRNA framework, on the one hand, and to the epsilon RNA, on the other hand, which shows that, in the chimeric RNA, each of the two constitutive parts retains its own structure. The numbering of the ribonucleotides in the HSQC spectrum corresponds to that given for the chimeric tRNA shown.

FIG. 4

FIG. 4 shows photographs of crystals of a chimeric tRNA^{Lys}₃ incorporating the epsilon domain of the human hepatitis B virus—Crystals obtained by sitting-drop vapour diffusion by means of the Natrix® kit (Hampton Research) on a CyBio HTPC crystallization robot. The drop represents a total volume of 1 μl.

FIG. 5

FIG. 5 shows the result of the digestion with RNase H of a chimeric tRNA^{Lys}₃ incorporating the epsilon domain of the human hepatitis B virus. The chimeric tRNA (approximately 50 μg) is hybridized with two DNA oligonucleotides complementary to the 5′ and 3′ regions of the epsilon RNA in a ratio 1:1:1 and then incubated at 37° C. in the presence of RNase H of E. coli (10 units/nmol DNA) in a buffer 100 mM NaCl, 5 mM MgCl₂, 50 mM Tris-HCl pH 7.5. Aliquots are removed at various incubation times and then analyzed by electrophoresis on 16% polyacrylamide-urea gel under denaturing conditions. The presence of RNA is visualized by ultraviolet (UV) shadowing. Left-hand lane: untreated chimeric tRNA. Right-hand lane: marker. In the centre, from left to right: points of the cleavage kinetics after incubation for 1 hour, 2 hours and 3 hours, respectively.

FIG. 6

FIG. 6 shows the result of a dimerization experiment on a chimeric tRNA incorporating the dimerization domain of the viral genomic RNA of HIV. The chimeric tRNA was incubated in the presence of 100 mM NaCl, 5 mM MgCl₂, 50 mM Tris-HCl pH 7.5 and then deposited on a non-denaturing 8% acrylamide gel (native conditions). Migration is effected at 4° C. in order to avoid fusion of the base pairings. The presence of RNA species is visualized by UV shadowing. Left-hand lane: control chimeric tRNA. Right-hand lane: chimeric tRNA carrying the HIV dimerization sequence. The two RNAs inserted into the chimeric tRNAs having the same size (approximately 110 ribonucleotides), the difference in migration under native conditions indicates the formation of the RNA dimer, via the viral RNA sequences.

FIG. 7

FIG. 7 shows the absorption spectrum of malachite green in the absence and in the presence of a chimeric tRNA incorporating an apatmer that binds to malachite green. The graph shows the absorption (Y-axis, arbitrary units) as a function of the wavelength (X-axis, in nm). The spectra are those of aqueous solutions of malachite green at the same concentration (approximately 100 nmol/liter), in the presence or in the absence of chimeric tRNA. In the presence of chimeric tRNA carrying the aptamer specific for the dye, a considerable increase in absorption and a shift of the maximum towards the red are observed. That displacement is not observed with a control chimeric tRNA. The phenomenon is analogous to that observed for the aptamer alone (without tRNA framework) and shows that its inclusion in the chimeric tRNA does not affect its functional properties.

FIG. 8

FIG. 8 shows the result of an experiment to determine the dissociation constant (Kd) between a chimeric tRNA incorporating an aptamer that binds to malachite green and malachite green. The curve shows the fluorescence signal (Y-axis, arbitrary units) emitted by the dye as a function of the concentration of chimeric tRNA added to the spectrophotofluorimeter vessel (X-axis, ×10⁻⁷ mol/liter) (excitation wavelength=610 nm, emisson wavelength=645 nm). The Kd is determined by iterative non-linear adjustment of the theoretical curve to the measured experimental values.

FIG. 9

FIG. 9 shows the structure of human chimeric tRNA^{Lys}₃ incorporating aptamers that bind to dextran (Sephadex™) (A) and to streptavidin (B) and of chimeric tRNAₘ^{Met} incorporating aptamers that bind to dextran (Sephadex™) (C) and to streptavidin (D).

FIG. 10

FIG. 10 shows the electrophoretic profile of the purification steps of a chimeric tRNA^{Lys}₃ incorporating an aptamer that binds to Sephadex™. The Sephadex™ beads were first equilibrated in a buffer A (Tris-HCl 50 mM pH 7.5; NaCl 100 mM; MgCl₂ 5 mM) and brought in the presence of total cell RNAs obtained by phenolic extraction, and then the whole was stirred for 30 minutes at 4° C. The beads were washed three times with buffer A and then the RNAs comprising an aptamer that binds to Sephadex™ were eluted with the aid of soluble dextran. The different fractions were then analyzed by electrophoresis on acrylamide-urea gel. From left to right: total RNAs, RNAs not retained on the beads, washings 1, 2 and 3, RNAs retained on the beads (before elution), RNAs eluted by soluble dextran.

FIG. 11

FIG. 11 shows the structure of a human chimeric tRNA^{Lys}₃ incorporating an aptamer that binds to streptavidin and to the epsilon domain of human HBV.

### EXAMPLES

### Example 1

### Production of a Chimeric tRNA Comprising the Epsilon Domain of the Hepatitis B Virus

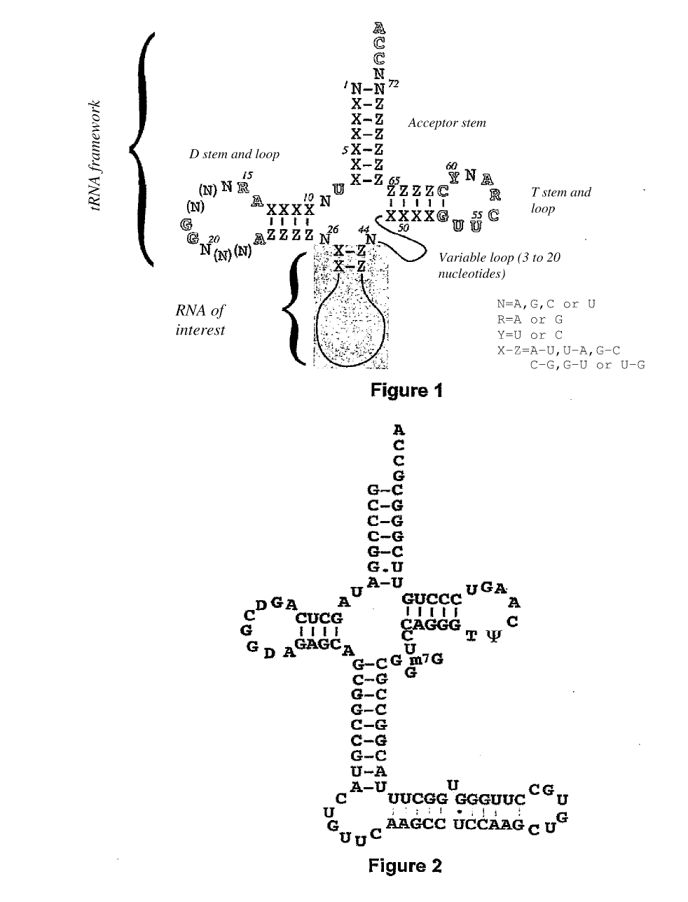

The human tRNA^{Lys}₃ was modified to incorporate the epsilon domain of the hepatitis B virus (FIG. 2).

Briefly, an expression vector pBSTNav-Lys comprising the coding sequence of human tRNA^{Lys}₃ modified by insertion of the restriction sites Eagl, EcoRV and SacII was prepared (SEQ ID NO: 7), and then the sequence of the epsilon domain of the human hepatitis B virus (SEQ ID NO: 17) was inserted between the sites Eagl and saclI to give the vector pBSTNav-Lys-epsilon.

That vector was used to transform E. coli bacteria. The bacteria were then cultured in a rich medium (Luria-Broth, LB), in the presence of ampicillin at a concentration of 100 μg/ml, for 14-15 hours at 37° C. The bacteria were recovered by centrifugation (30 minutes at 4000 rpm for 1 liter of culture). The pellet was suspended in 8.6 ml of buffer 10 mM Mg acetate, 10 mM Tris-HCl pH 7.4. 10 ml of saturated phenol in the same buffer were then added, and the whole was stirred gently for one hour at room temperature and then centrifuged for 30 minutes at 10,000 rpm. 0.1 volume of NaCl 5M and 2 volumes of pure ethanol were then added to the aqueous phase. The whole was centrifuged for 30 minutes at 10,000 rpm (4° C.), and the pellet was recovered and suspended in 5 ml of NaCl 1M. The solubilisate was centrifuged for 30 minutes at 10,000 rpm (4° C.), and then the supernatant was recovered. 2.5 volumes of ethanol were then added, and centrifugation was again carried out for 30 minutes at 10,000 rpm (4° C.). The pellet was recovered and dissolved in water. From one liter of culture in LB (Luria-Broth) medium, approximately 100 mg of total RNA are obtained.

In some cases, the tRNAs were then purified on anion-exchange resin (60 ml of phase, Q-sepharose, Pharmacia). The purification was carried out in sodium phosphate pH 6.5 50 mM, with a gradient going from 500 mM to 650 mM of NaCl on 475 ml with a flow rate of 0.5 ml/minute. The chimeric tRNA is eluted after the shorter, endogenous tRNAs. After this step, starting from 1 liter of culture, at the end of ion exchange, approximately 50 mg of purified chimeric tRNA (tRNA^{Lys}₃+epsilon) are obtained.

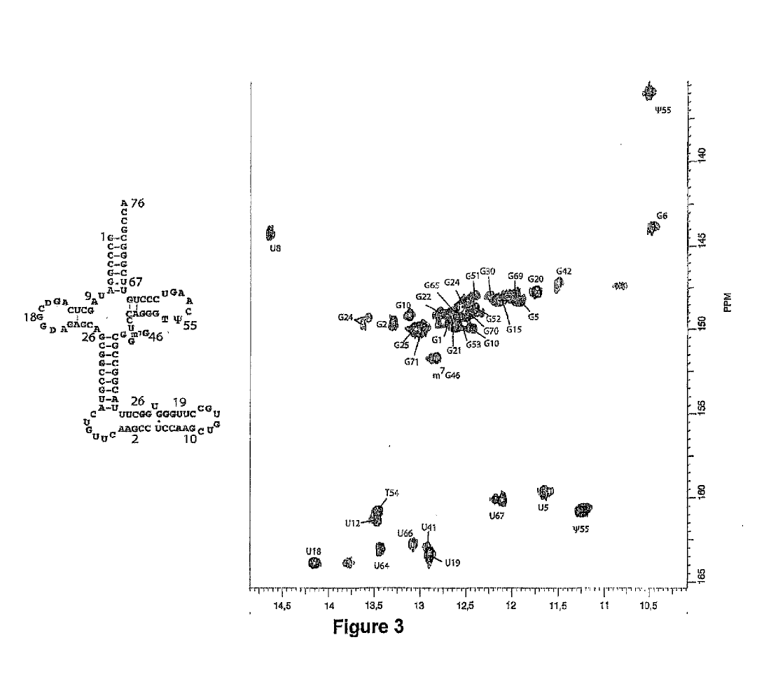

The chimeric tRNA^{Lys}₃ comprising the epsilon domain of the hepatitis B virus labelled with ¹⁵N nitrogen by applying the procedure described above to a bacteria culture cultivated on an enriched medium (Spectra-9N medium, Spectra Stable Isotopes, or equivalent) was characterized by nuclear magnetic resonance (NMR). The HSQC (heteronuclear single-quantum correlation ¹H-¹⁵N) spectrum shown in FIG. 3 thus shows that the epsilon domain is structured according to its natural conformation. This demonstrates that the method for producing RNA according to the invention allows correctly structured RNAs to be obtained and, moreover, that the chimeric tRNAs according to the invention can be used for the resolution of NMR structures of RNA molecules without it being necessary to separate them from the chimeric tRNA.

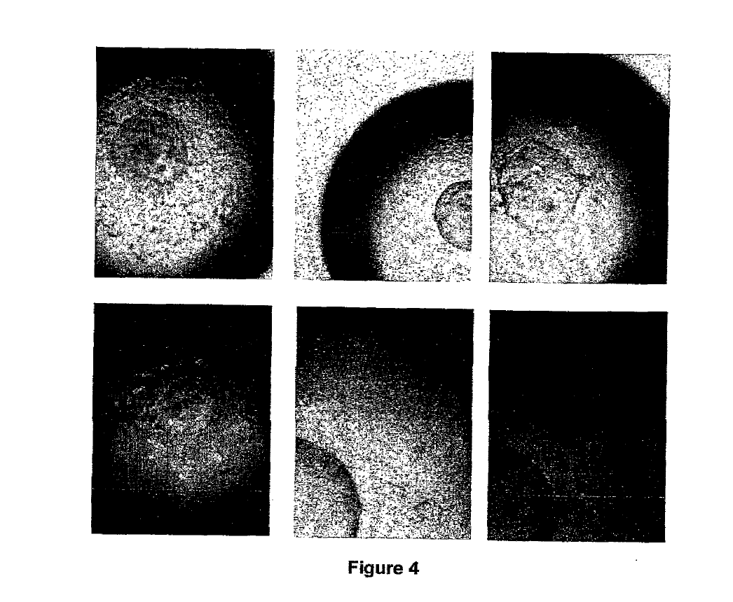

In addition, it was possible to crystallize the chimeric tRNA^{Lys}₃ comprising the epsilon domain of the hepatitis B virus (FIG. 4). This also demonstrates the interest of the method for producing RNA according to the invention for the resolution of crystallographic structures. That interest is reinforced by the fact that, where the crystallographic structure of the tRNA from which the chimeric tRNA is formed is known, it is then possible to use that structure for the step of framing or molecular replacement during the resolution of the crystallographic structure of the chimeric tRNA as a whole.

Finally, the epsilon domain of the hepatitis B virus was separated from the chimeric tRNA^{Lys}₃ by digestion with RNase H.

Briefly, two oligonucleotides (SEQ ID NO: 13 and 14) in aqueous solution are brought in the presence of the chimeric tRNA^{Lys3} in a molar ratio 1:1. The mixture so obtained (approximately 100 μl) is brought to 95° C. in a water bath and then, after cooling to room temperature, a buffer is added in order to obtain, in final concentration, 100 mM NaCl, 5 mM MgCl₂, 50 mM Tris-HCl pH 7.5 and RNase H of E. coli(10 U/nmol of DNA), which is allowed to act for 4 hours at 37° C. The result of the digestion is shown in FIG. 5.

In the same manner as above, an expression vector pBSTNav-Met comprising the coding sequence of the tRNAtₘ^{Met} of Escherichia Coli modified by insertion of the restriction sites Eagl, EcoRV and SacII was prepared (SEQ ID NO: 8), and then the sequence of the epsilon domain of the hepatitis B virus (SEQ ID NO 17) was inserted between the sites Eagl and SacII to give the vector pBSTNav-Met-epsilon.

### Example 2

### Production of a Chimeric tRNA Comprising the Genomic Dimerization Site of the Human Immunodeficiency Virus (HIV)

The genomic dimerization site of HIV was inserted into the human tRNA^{Lys}₃ or the tRNAₘ^{Met} of Escherichia Coli, as described in Example 1, by insertion of a DNA encoding the dimerization site (SEQ ID NO: 18) into the expression vectors pBSTNav-Lys (SEQ ID NO: 7) and pBSTNav-Met (SEQ ID NO: 8) respectively, after cleavage by the restriction enzymes Eagl and SacII. The method for the production of the corresponding chimeric tRNA and the yields are analogous to those of Example 1.

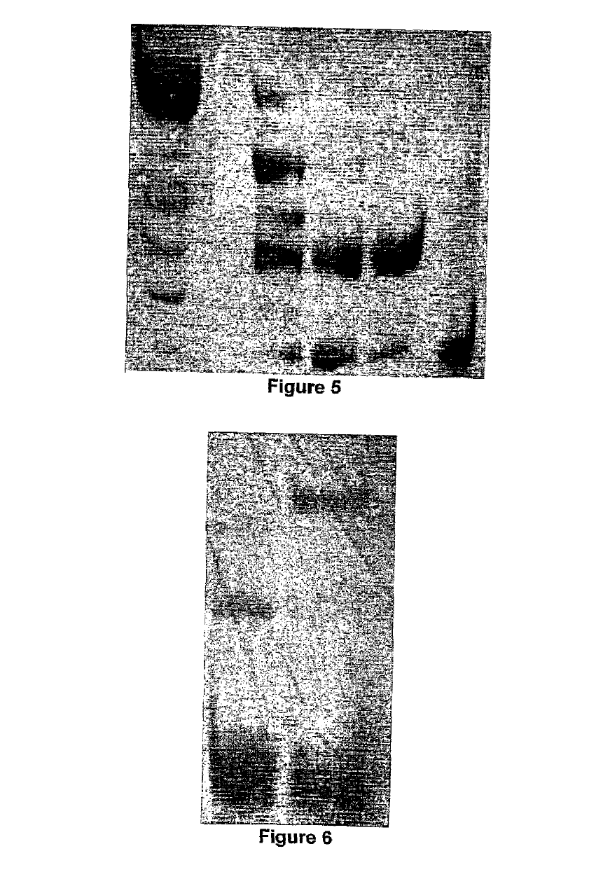

The functionality of the dimerization site of the genomic RNA of HIV within the framework formed by the tRNA was checked by electrophoresis in native 8% acrylamide gel in the absence of urea, at 4° C. (FIG. 6). Under those conditions, migration of a species corresponding to double the expected size for the chimeric tRNA is observed, which demonstrates the formation of the dimer and, therefore, the functional nature of the viral RNA sequence inserted into the tRNA framework.

### Example 3

Production of a Chimeric tRNA Comprising an Aptamer that Binds to Malachite Green

An aptamer that binds to malachite green was inserted into human tRNA^{Lys}₃, as described in Example 1, by insertion of a DNA encoding the aptamer (SEQ ID NO: 19) into the expression vector pBSTNav-Lys (SEQ ID NO: 7) after cleavage by the restriction enzymes Eagl and SacII. The method for the production of the corresponding chimeric tRNA and the yields are analogous to those of Example 1.

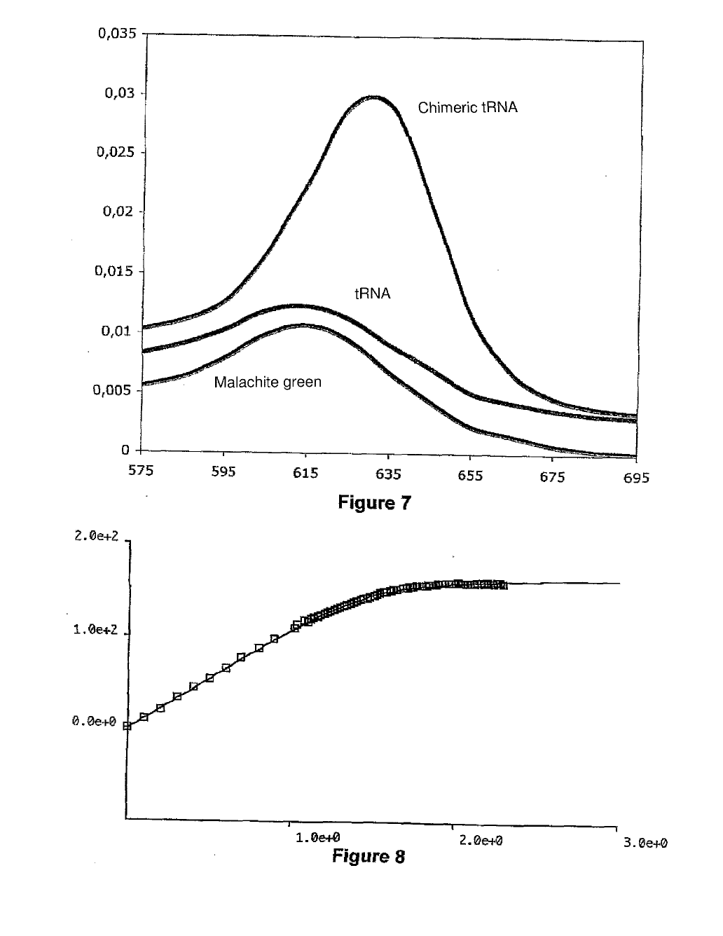

The functionality of the aptamer was checked by verifying that the chimeric tRNA was capable of binding malachite green—the binding of the dye to the aptamer manifesting itself in an increase in its molar extinction coefficient (FIG. 7). The dissociation constant of the chimeric tRNA according to the invention for malachite green was estimated at 50.10⁻⁹ mol/liter (FIG. 8), which is similar to the value measured for the aptamer alone. Advantageously, the chimeric tRNAs comprising an aptamer according to the invention can therefore be used directly as an aptamer, without it being necessary to cleave the tRNA framework.

### Example 4

### Production of a Chimeric tRNA Comprising a Portion of the 16S rRNA of E Coli

A part of the 16S rRNA of E. coli was inserted into the human tRNA^{Lys}₃ as described in Example 1, by insertion of a DNA coding for the portion of rRNA (SEQ ID NO: 20) into the expression vector pBSTNav-Lys (SEQ ID NO: 7) after cleavage by the restriction enzymes Eagl and SacII.

That chimeric tRNA can be used, for example, for screening antibiotic compounds that act on that region of the bacterial ribosome, such as, for example, aminoglycosides and their analogs.

### Example 5

### Production of a Chimeric tRNA Comprising an Aptamer that Binds to Streptavidin or to Sephadex™

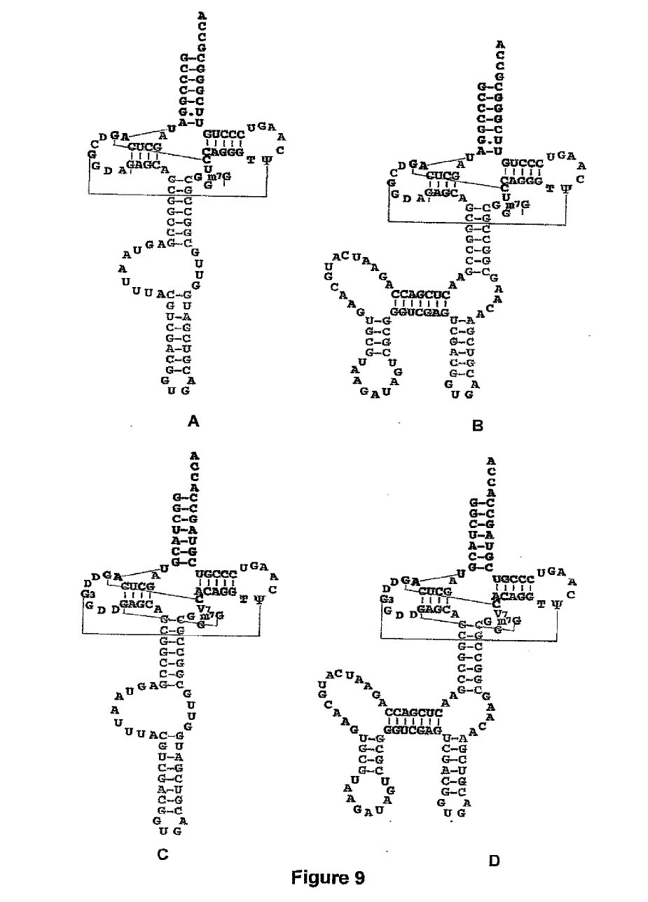

An aptamer that binds to streptavidin was inserted into the human tRNA^{Lys}₃ or the tRNAₘ^{Met} of Escherichia Coli, as described in Example 1, by insertion of a DNA encoding the aptamer (SEQ ID NO: 21) into the expression vector pBSTNav-Lys (SEQ ID NO: 7) and pBSTNav-Met (SEQ ID NO: 8) after cleavage by the restriction enzymes Eagl and SacII, to give pBSTNav-Lys-strepta and pBSTNav-Met-strepta (see FIG. 9).

Likewise, an aptamer that binds to Sephadex™ (beads of dextran derivative marketed by Pharmacia) was inserted into the human tRNA^{Lys}₃ or the tRNAₘ^{Met} of Escherichia Coli, as described in Example 1, by insertion of a DNA encoding the aptamer (SEQ ID NO: 22) into the expression vector pBSTNav-Lys (SEQ ID NO: 7) and pBSTNav-Met (SEQ ID NO: 8) respectively, after cleavage by the restriction enzymes Eagl and SacII, to give pBSTNav-Lys-sepha and pBSTNav-Met-sepha (see FIG. 9).

The functionality of the tRNA^{Lys}₃ comprising an aptamer that binds to Sephadex™ is shown by way of example. To that end, a solution of RNA obtained after extraction with phenol, as shown in Example 1, was purified directly with the aid of Sephadex™ beads.

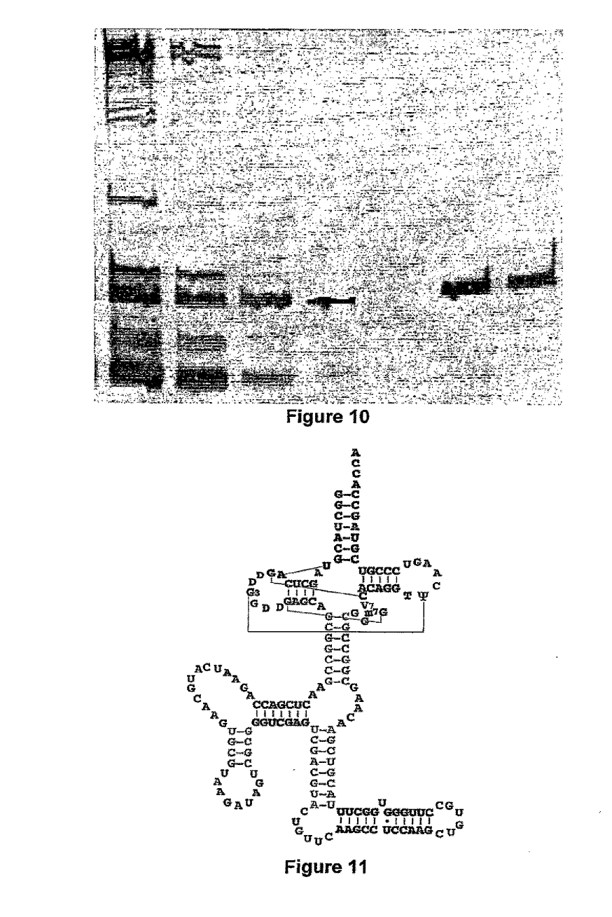

Briefly, the Sephadex™ beads were first equilibrated in a buffer A (Tris-HCl 50 mM pH 7.5; NaCl 100 mM; MgCl₂ 5 mM) and brought in the presence of the RNAs, and then the whole was stirred for 30 minutes at 4° C. The beads were washed three times with buffer A and then the RNAs comprising an aptamer that binds to Sephadex™ were eluted with the aid of soluble dextran (Sigma-Aldrich). The results of this purification are shown in FIG. 10.

In addition, two restriction sites SalI and AatlI were inserted into the part corresponding to the sequence coding for the aptamer, permitting the provision of a complete system for production and purification of RNA, constituted by the vectors pBSTNav-Lys-sepha, pBSTNav-Met-sepha, pBSTNav-Lys-strepta and pBSTNav-Met-strepta (SEQ ID NO: 9 to 12).

A plurality of RNAs have thus been expressed with the aid of that system, especially the epsilon domain of the human hepatitis B virus (HBV) (SEQ ID NO: 23) (FIG. 11), the epsilon domain of duck HBV (SEQ ID NO: 24), and the domain of interaction of 23S ribosomal RNA with the ribosomal protein L20 (SEQ ID NO: 25), the latter RNA being useful especially for screening antibiotics directed against the bacterial ribosome.

1-27. (canceled)

28. A method for the production of a RNA, wherein:
prokaryotic cells transformed by a nucleic acid coding for a chimeric transfer RNA (tRNA), which chimeric tRNA is derived from the modification of a tRNA by insertion of a RNA into the stem-loop of the anticodon of said tRNA and/or by substitution of all or part of the stem-loop of the anticodon of said tRNA with a are cultivated;
the chimeric tRNA is recovered from the cultivated cells or from the culture supernatant of the cultivated cells;
the chimeric tRNA is optionally cleaved in order to recover the RNA to be produced in isolated form.

29. The method according to claim 28, wherein the RNA substitutes all or part of the stem-loop of the anticodon contained between the first ribonucleotide, inclusive, of the stem-loop of the anticodon and the last ribonucleotide, inclusive, of the stem-loop of the anticodon.

30. The method according to claim 28, wherein the chimeric tRNA has the following formula (I): 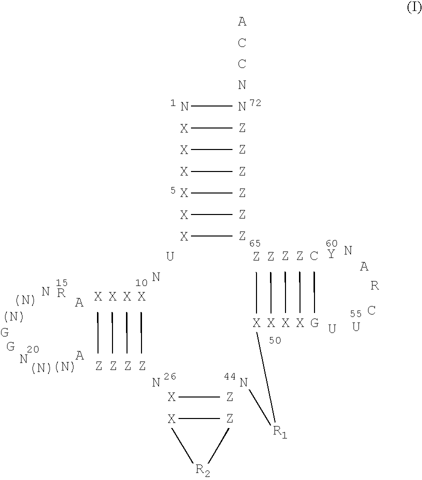
wherein:
A represents adenine or one of its analogs, C represents cytosine or one of its analogs, G represents guanosine or one of its analogs, and U represents uridine or one of its analogs,
each of the N, which may be identical or different, represents any ribonucleotide,
each of the (N), which may be identical or different, represents any ribonucleotide, which may be present or absent,
R represents A or G, or their analogs,
Y represents U or C, or their analogs,
each of the X-Z, which may be identical or different, represents a pair A-U, U-A, G-C, C-G, G-U or U-G, or their analogs,
the ribonucleotides N in position 1 and N in position 72 may or may not be paired,
R₁ represents a sequence of from 3 to 20 ribonucleotides,
R₂ represents the inserted RNA, namely a sequence of from 6 to 300 ribonucleotides.

31. The method according to claim 28, wherein the tRNA is selected from the group constituted by Archean, bacterial, viral, protozoan, fungal, algal, plant and animal tRNAs.

32. The method according to claim 28, wherein all or part of the RNA is selected from the list constituted by an antisense RNA, an interfering RNA, an aptamer, a ribozyme, a viral RNA, a ribosomal RNA and a nucleolar RNA.

33. The method according to claim 28, wherein the RNA is structured.

34. The method according to claim 28, wherein the RNA comprises a purification tag.

35. The method according to claim 34, wherein the purification tag is selected from the group constituted by a ribozyme and an aptamer.

36. The method according to claim 28, wherein the chimeric tRNA has one of the following formulae: 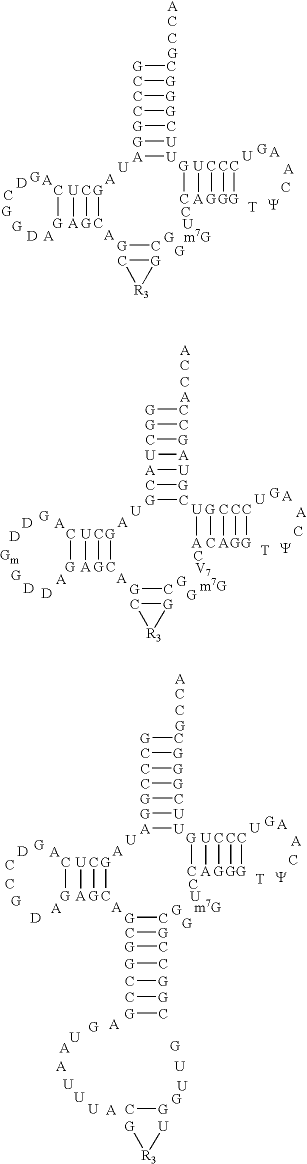 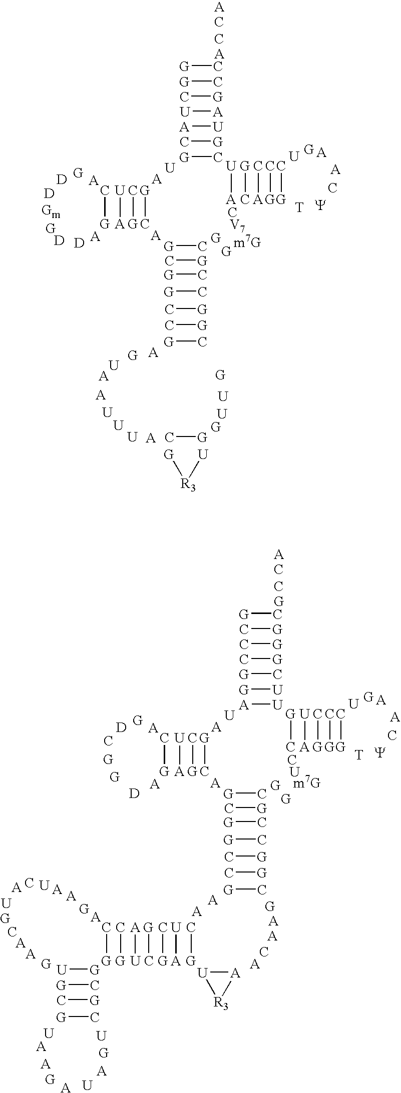 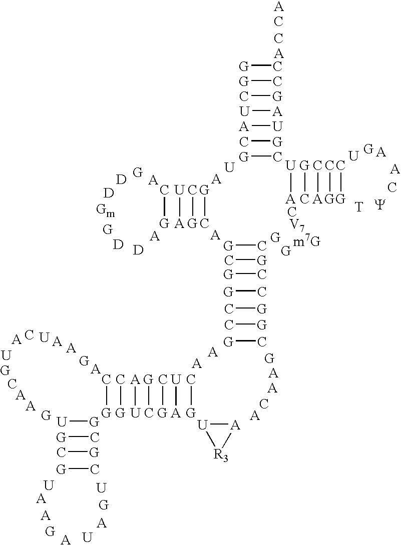
wherein:
D represents dihydrouridine,
T represents ribothymidine,
Φ represents pseudouridine,
^{m7}G represents 7-methyl-guanine,
V₇ represents 3-(3-amino-3-carboxypropyl)-uridine,
R₃ represents a sequence of from 6 to 300 ribonucleotides.

37. The method according to claim 28, wherein the nucleic acid is contained in an expression vector comprising a promoter and a terminator, which are operably linked to the nucleic acid, as well as a replication origin and a selection marker.

38. The method according to claim 28, wherein the chimeric tRNA does not comprise the substantially intact stem of the anticodon of the tRNA from which it is derived.

39. A chimeric tRNA which chimeric tRNA is derived from the modification of a tRNA by insertion of a RNA into the stem-loop of the anticodon of said tRNA and/or by substitution of all or part of the stem-loop of the anticodon of said tRNA with a RNA, wherein the chimeric tRNA does not comprise the substantially intact stem of the anticodon of the tRNA from which it is derived.

40. A nucleic acid coding for a chimeric tRNA, which chimeric tRNA is derived from the modification of a tRNA by insertion of a RNA into the stem-loop of the anticodon of said tRNA and/or by substitution of all or part of the stem-loop of the anticodon of said tRNA with a RNA, wherein the chimeric tRNA does not comprise the substantially intact stem of the anticodon of the tRNA from which it is derived.

41. An expression vector comprising a nucleic acid as defined in claim 40, a promoter and a terminator, which are operably linked to the nucleic acid, as well as a replication origin and a selection marker.

42. A cell comprising a nucleic acid as defined in claim 40, or an expression vector comprising said nucleic acid, said expression also comprising a promoter and terminator, which are operably linked to the nucleic acid, as well as a replication origin and a selection marker.

43. A nucleic acid suitable for the preparation of a nucleic acid coding for a chimeric tRNA, which chimeric tRNA is derived from the modification of a tRNA by insertion of a RNA into the stem-loop of the anticodon of said tRNA and/or by substitution of all or part of the stem-loop of the anticodon of said tRNA with a RNA, wherein the chimeric tRNA does not comprise the substantially intact stem of the anticodon of the tRNA from which it is derived, said nucleic acid comprising, in the 5′-3′ direction, at least:
(i) a sequence coding for a part of a tRNA extending from the 5′ end of said tRNA to the ribonucleotide that precedes the first ribonucleotide of the stem of the anticodon or to a ribonculeotide of the stem or of the loop of the anticodon;
(ii) optionally a sequence coding for all or part of a purification tag;
(iii) at least one cleavage site of a restriction enzyme;
(iv) optionally a sequence coding for all or part of a purification tag;
(v) a sequence coding for a part of the tRNA extending from a ribonucleotide of the stem or of the loop of the anticodon downstream of the ribonucleotide of (i) or of the ribonucleotide that follows the last ribonucleotide of the stem of the anticodon to the 3′ end of said tRNA,
provided that the sequence of the second nucleic acid in its entirety is not the coding sequence of the tRNA.

44. The nucleic acid according to claim 43, the sequence of which is selected from the group constituted by:
SEQ ID NO: 1;
SEQ ID NO: 2;
SEQ ID NO: 3;
SEQ ID NO: 4;
SEQ ID NO: 5;
SEQ ID NO: 6.

45. An expression vector comprising a nucleic acid as defined in claim 43, a promoter and a terminator, which are operably linked to the nucleic acid, as well as a replication origin and a selection marker.

46. The expression vector according to claim 45, the sequence of which is selected from the group constituted by:
SEQ ID NO: 7;
SEQ ID NO: 8;
SEQ ID NO: 9;
SEQ ID NO: 10;
SEQ ID NO: 11;
SEQ ID NO: 12.

47. A kit for the production of a RNA with the aid of a chimeric tRNA comprising it, which kit comprises at least:
an expression vector as defined in claim 18;
a means for cleaving a chimeric tRNA allowing to release the RNA to be produced;
optionally at least one restriction enzyme that cleaves at least one cleavage site of said restriction enzyme;
optionally cells capable of being transformed by a nucleic acid and of producing a chimeric tRNA.

48. The kit according to claim 47, wherein the expression vector is a bacterial plasmid and the cells are bacteria.

49. The kit according to claim 47, wherein the cleavage means is constituted by RNase H and two oligonucleotides that are complementary, respectively, to part of the sequence of the chimeric tRNA that precedes the 5′ end of the RNA to be produced, and to part of the sequence of the chimeric tRNA that follows the 3′ end of the RNA to be produced.

50. The kit according to claim 47, wherein the cleavage means is constituted by RNase H and two oligonucleotides that are complementary, respectively, to part of the sequence of the chimeric tRNA that precedes the 5′ end of the RNA to be produced, and to part of the sequence of the chimeric tRNA that follows the 3′ end of the RNA to be produced, and wherein:
the bacteria are of the Escherichia coli type;
the expression vector is represented by SEQ ID NO: 7 and the oligonucleotides are represented by SEQ ID NO: 13 and SEQ ID NO: 14, or
the expression vector is represented by SEQ ID NO: 8 and the oligonucleotides are represented by SEQ ID NO: 15 and SEQ ID NO: 16, or
the expression vector is represented by SEQ ID NO: 9 and the oligonucleotides are represented by SEQ ID NO: 13 and SEQ ID NO: 14, or
the expression vector is represented by SEQ ID NO 10 and the oligonucleotides are represented by SEQ ID NO: 15 and SEQ ID NO: 16, or
the expression vector is represented by SEQ ID NO 11 and the oligonucleotides are represented by SEQ ID NO: 13 and SEQ ID NO: 14, or
the expression vector is represented by SEQ ID NO: 12 and the oligonucleotides are represented by SEQ ID NO: 15 and SEQ ID NO: 16.

51. A method for resolving the three-dimensional structure of a RNA inserted or substituted in a chimeric tRNA as defined in claim 28 comprising applying the technique of nuclear magnetic resonance to a solution of said chimeric tRNA, wherein the RNA to be studied is inserted or substituted, or applying the technique of X-ray diffraction to crystals of said chimeric tRNA, and deducing therefrom the three-dimensional structure of said RNA.

52. A method for preventing or limiting the expression of a target gene comprising introducing into a cell a chimeric tRNA as defined in claim 28 wherein the inserted or substituted RNA is an interfering RNA, and allowing the cell to produce said RNA.

53. A method for binding a target compound comprising introducing into a cell a chimeric tRNA as defined in claim 28 wherein the inserted or substituted RNA is an aptamer, and allowing the cell to produce said RNA.

54. A pharmaceutical composition comprising as active ingredient at least one chimeric tRNA as defined in claim 38, in association with a pharmaceutically acceptable carrie


SEQUENCE LISTING (25 sequences)

SEQ ID NO: 1 - DNA, 74 bp, Artificial sequence
  synthetic Sequence
gcccggatagctcagtcggtagagcagcggccgatatccgcgggtccagggttcaagtccctgttcgggcgcca

SEQ ID NO: 2 - DNA, 75 bp, Artificial sequence
  synthetic Sequence
ggctacgtagctcagttggttagagcagcggccgatatccgcgggtcacaggttcgaatcccgtcgtagccacca

SEQ ID NO: 3 - DNA, 106 bp, Artificial sequence
  synthetic Sequence
gcccggatagctcagtcggtagagcagcggccgagtaatttacgtcgacggtgacgtcgatggttgcggccgcgggtccagggttcaagtccctgttcgggcgcca

SEQ ID NO: 4 - DNA, 107 bp, Artificial sequence
  synthetic Sequence
ggctacgtagctcagttggttagagcagcggccgagtaatttacgtcgacggtgacgtcgatggttgcggccgcgggtcacaggttcgaatcccgtcgtagccacca

SEQ ID NO: 5 - DNA, 140 bp, Artificial sequence
  synthetic Sequence
gcccggatagctcagtcggtagagcagcggccgaactcgaccagaatcatgcaagtgcgtaagatagtcgcgggtcgagtcgacggtgacgtcgaacaagcggccgcgggtccagggttcaagtccctgttcgggcgcca

SEQ ID NO: 6 - DNA, 141 bp, Artificial sequence
  synthetic Sequence
ggctacgtagctcagttggttagagcagcggccgaactcgaccagaatcatgcaagtgcgtaagatagtcgcgggtcgagtcgacggtgacgtcgaacaagcggccgcgggtcacaggttcgaatcccgtcgtagccacca

SEQ ID NO: 7 - DNA, 3087 bp, Artificial sequence
  synthetic Sequence
ggaaacagctatgaccatgattacgccaagctcggaattaaccctcactaaagggaacaaaagctgggtaccgggccccccctcgaggtcgccccatcaaaaaaatattctcaacataaaaaactttgtgtaatacttgtaacgctgaattcgcccggatagctcagtcggtagagcagcggccgatatccgcgggtccagggttcaagtccctgttcgggcgccactgcagatccttagcgaaagctaaggattttttttaagcttgatgggggcttaggatccggagctccaagctttagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaacttgaacaacactcaaccctatctcgggctattcttttgatttataagggattttgccgatttcggcctattggttaaaaaatgagctgatttaacaaaaatttaacgcgaattttaacaaaatattaacgtttacaatttaaatcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgccagcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacaca

SEQ ID NO: 8 - DNA, 3088 bp, Artificial sequence
  synthetic Sequence
ggaaacagctatgaccatgattacgccaagctcggaattaaccctcactaaagggaacaaaagctgggtaccgggccccccctcgaggtcgccccatcaaaaaaatattctcaacataaaaaactttgtgtaatacttgtaacgctgaattcggctacgtagctcagttggttagagcagcggccgatatccgcgggtcacaggttcgaatcccgtcgtagccaccactgcagatccttagcgaaagctaaggattttttttaagcttgatgggggcttaggatccggagctccaagctttagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaacttgaacaacactcaaccctatctcgggctattcttttgatttataagggattttgccgatttcggcctattggttaaaaaatgagctgatttaacaaaaatttaacgcgaattttaacaaaatattaacgtttacaatttaaatcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgccagcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacaca

SEQ ID NO: 9 - DNA, 3119 bp, Artificial sequence
  synthetic Sequence
ggaaacagctatgaccatgattacgccaagctcggaattaaccctcactaaagggaacaaaagctgggtaccgggccccccctcgaggtcgccccatcaaaaaaatattctcaacataaaaaactttgtgtaatacttgtaacgctgaattcgcccggatagctcagtcggtagagcagcggccgagtaatttacgtcgacggtgacgtcgatggttgcggccgcgggtccagggttcaagtccctgttcgggcgccactgcagatccttagcgaaagctaaggattttttttaagcttgatgggggcttaggatccggagctccaagctttagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaacttgaacaacactcaaccctatctcgggctattcttttgatttataagggattttgccgatttcggcctattggttaaaaaatgagctgatttaacaaaaatttaacgcgaattttaacaaaatattaacgtttacaatttaaatcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgccagcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacaca

SEQ ID NO: 10 - DNA, 3120 bp, Artificial sequence
  synthetic Sequence
ggaaacagctatgaccatgattacgccaagctcggaattaaccctcactaaagggaacaaaagctgggtaccgggccccccctcgaggtcgccccatcaaaaaaatattctcaacataaaaaactttgtgtaatacttgtaacgctgaattcggctacgtagctcagttggttagagcagcggccgagtaatttacgtcgacggtgacgtcgatggttgcggccgcgggtcacaggttcgaatcccgtcgtagccaccactgcagatccttagcgaaagctaaggattttttttaagcttgatgggggcttaggatccggagctccaagctttagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaacttgaacaacactcaaccctatctcgggctattcttttgatttataagggattttgccgatttcggcctattggttaaaaaatgagctgatttaacaaaaatttaacgcgaattttaacaaaatattaacgtttacaatttaaatcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgccagcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacaca

SEQ ID NO: 11 - DNA, 3153 bp, Artificial sequence
  synthetic Sequence
ggaaacagctatgaccatgattacgccaagctcggaattaaccctcactaaagggaacaaaagctgggtaccgggccccccctcgaggtcgccccatcaaaaaaatattctcaacataaaaaactttgtgtaatacttgtaacgctgaattcgcccggatagctcagtcggtagagcagcggccgaactcgaccagaatcatgcaagtgcgtaagatagtcgcgggtcgagtcgacggtgacgtcgaacaagcggccgcgggtccagggttcaagtccctgttcgggcgccactgcagatccttagcgaaagctaaggattttttttaagcttgatgggggcttaggatccggagctccaagctttagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaacttgaacaacactcaaccctatctcgggctattcttttgatttataagggattttgccgatttcggcctattggttaaaaaatgagctgatttaacaaaaatttaacgcgaattttaacaaaatattaacgtttacaatttaaatcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgccagcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacaca

SEQ ID NO: 12 - DNA, 3154 bp, Artificial sequence
  synthetic Sequence
ggaaacagctatgaccatgattacgccaagctcggaattaaccctcactaaagggaacaaaagctgggtaccgggccccccctcgaggtcgccccatcaaaaaaatattctcaacataaaaaactttgtgtaatacttgtaacgctgaattcggctacgtagctcagttggttagagcagcggccgaactcgaccagaatcatgcaagtgcgtaagatagtcgcgggtcgagtcgacggtgacgtcgaacaagcggccgcgggtcacaggttcgaatcccgtcgtagccaccactgcagatccttagcgaaagctaaggattttttttaagcttgatgggggcttaggatccggagctccaagctttagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaacttgaacaacactcaaccctatctcgggctattcttttgatttataagggattttgccgatttcggcctattggttaaaaaatgagctgatttaacaaaaatttaacgcgaattttaacaaaatattaacgtttacaatttaaatcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgccagcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacaca

SEQ ID NO: 13 - DNA, 15 bp, Artificial sequence
  synthetic Sequence
gaaccctggacccgc

SEQ ID NO: 14 - DNA, 15 bp, Artificial sequence
  synthetic Sequence
gctgctctaccgact

SEQ ID NO: 15 - DNA, 16 bp, Artificial sequence
  synthetic Sequence
gaacctgtgacccagc

SEQ ID NO: 16 - DNA, 15 bp, Artificial sequence
  synthetic Sequence
gctgctcaaccaact

SEQ ID NO: 17 - DNA, 51 bp, Artificial sequence
  synthetic Sequence
ggcccgtgtatctttacgtctacattgctgttgtcgtgtgtgactgtacgc

SEQ ID NO: 18 - DNA, 43 bp, Artificial sequence
  synthetic Sequence
ggcctcggcttgctgaggtgcacacagcaagaggcgaggccgc

SEQ ID NO: 19 - DNA, 44 bp, Artificial sequence
  synthetic Sequence
ggccatcccgactggcgagagccaggtaacgaatggatggccgc

SEQ ID NO: 20 - DNA, 33 bp, Artificial sequence
  synthetic Sequence
ggcccgtcacaccttcgggtgaagtcgggccgc

SEQ ID NO: 21 - DNA, 79 bp, Artificial sequence
  synthetic Sequence
ggccgaactcgaccagaatcatgcaagtgcgtaagatagtcgcgggtcgagtcgacggtgacgtcgaacaagcggccgc

SEQ ID NO: 22 - DNA, 44 bp, Artificial sequence
  synthetic Sequence
ggccgagtaatttacgtcgacgtgacgtcgatggttgcggccgc

SEQ ID NO: 23 - DNA, 53 bp, Artificial sequence
  synthetic Sequence
tcgacgtgtatctttacgtctacattgctgttgtcgtgtgtgactgtacacgt

SEQ ID NO: 24 - DNA, 48 bp, Artificial sequence
  synthetic Sequence
tcgacgtactgttcaagcctccaagctgtgccttgggtggctttacgt

SEQ ID NO: 25 - DNA, 53 bp, Artificial sequence
  synthetic Sequence
tcgacgtcagctaaggtcccaaagtcatgggaaaccatgcaccgaagctacgt